(19) United States
(12) Patent Application Publication
(10) Pub. No.: US 2012/0122825 A1
(43) Pub. Date: May 17, 2012

(54) USE OF PICOPLATIN TO TREAT PROSTATE CANCER

(75) Inventors: David A. Karlin MD, Los Altos, CA (US); Hazel B. Breitz MD, Seattle, WA (US); Paul L. Weiden MD, Seattle, WA (US); Robert De Jager, Rancho Santa Fe, CA (US)

(73) Assignee: Poniard Pharmaceuticals, Inc., Seattle, WA (US)

(21) Appl. No.: 13/319,647

(22) PCT Filed: May 12, 2010
(86) PCT No.: PCT/US10/34591

§ 371 Date: Jan. 30, 2012

Related U.S. Application Data
(60) Provisional application No. 61/177,567, filed on May 12, 2009.

(51) Int. Cl. A61K 31/555; A61P 25/00; A61P 35/00; A61K 31/573; A61P 29/00

(52) U.S. Cl. 514/171; 514/188

(57) ABSTRACT

The invention provides a method of treatment of metastatic hormone-refractory prostate cancer involving substantially as concurrent administration of picoplatin and docetaxel. Prednisone may also be administered. Dosages and dosing regimens are provided.

### CROSS-REFERENCE TO RELATED APPLICATIONS

This application claims priority of U.S. provisional application Ser. No. 61/177,567, filed May 12, 2009, which is incorporated by reference herein.

### BACKGROUND OF THE INVENTION

Prostate cancer is diagnosed in approximately 220,000 men in the United States annually, and about 29,000 men died of the disease in 2004 (American Cancer Society, 2004). Patients with early disease are usually offered potentially curative treatment (radiotherapy, radical prostatectomy) and may also receive adjuvant hormone treatment. However, many of these patients will develop local recurrence and/or metastatic disease (Oh, Hurwitz, 2003). For patients with newly diagnosed metastatic disease, androgen suppression is the standard treatment; however, relapse usually occurs due to the development of androgen resistance, and the majority of patients develop hormone refractory prostate cancer (HRPC) after a median time of 18 to 24 months (Mahler, 1995; Kasamon, 2004; Cho, 2003; Rini, 2002).

Metastatic HRPC may be treated with cytotoxic therapies; however, until recently, most clinical trials using cytotoxic agents resulted in few objective responses, and no convincing improvement in survival. They have, however, indicated that improvements in serum PSA levels, quality of life, and symptom control can be achieved (Dowling, 2001; Tannock, 1996; Ernst, 2003).

In 2004, two randomized Phase 3 studies were reported that have changed the standard of care for fit men with HRPC (Tannock, 2004; Petrylak, 2004). Tannock, et al. assigned 1006 men with HRPC to receive 5 mg prednisone twice daily and either 12 mg/m² of mitoxantrone every 3 weeks, 75 mg/m² docetaxel every 3 weeks, or 30 mg/m² docetaxel weekly for 5 of 6 weeks. The men who received docetaxel every 3 weeks had significantly improved survival, PSA levels, pain levels, and quality of life when compared with the group that received mitoxantrone. Overall survival in the group receiving mitoxantrone was 16.5 months, in the group receiving docetaxel every 3 weeks was 18.9 months, and in the group receiving weekly docetaxel was 17.4 months, and >50% reductions in PSA were observed in 32%, 45%, and 48% of men in these groups, respectively. Adverse events (AEs), albeit more common in the patients who received docetaxel, did not prevent most patients from receiving their intended dose of the assigned drug on schedule.

In the second study, Petrylak, et al. (2004) assigned 770 men with HRPC to receive either estramustine 280 mg three times daily for Days 1 through 5 and 60 mg/m² docetaxel preceded by 60 mg of dexamethasone on Day 2 of a 21-day cycle, or 12 mg/m² of mitoxantrone on Day 1 plus 5 mg prednisone twice daily for each 21-day cycle. The median overall survival was 17.5 months in the group that received estramustine plus docetaxel and was 15.6 months in the group that received mitoxantrone and prednisone (p=0.002). PSA declines of >50% were reported in 50% and 27% of patients, respectively (p<0.001). Improved pain relief was not noted with docetaxel treatment in this study. Similar results have also been reported by Oudard, et al. (2005) in a randomized Phase 2 trial comparing docetaxel, estramustine, and prednisone to mitoxantrone and prednisone in a total of 130 patients.

Thus, while neither the design nor the results of these three studies were identical, they all demonstrated a statistically and clinically significant prolongation of survival in men with HRPC who received docetaxel compared to those who did not. This increase in survival was associated with improvements in pain and quality of life and with acceptable toxicity, thus establishing, in the opinion of most physicians and patients, a new standard of care. At the same time, the benefit of docetaxel is measured in months with no suggestion of curative potential. Thus there remains a clear need for additional improvements in the treatment of men with HRPC. Current FDA-approved platinum agents have limited activity in HRPC, when used either as monotherapy or in combination with other chemotherapy agents. In single agent studies, response rates of 0-29% have been reported (Rosoff, 1979; Merrin, 1979; Loening, 1983; Soloway, 1983; Canobbio, 1993) but overall clinical benefit seemed modest, especially compared to the toxicities observed in this patient population. These included, in varying degree, nausea and vomiting, myelosuppression, and of perhaps greatest impact, significant nephro-, neuro-, and ototoxicity (Reed, 1993). Platinum compounds, either carboplatin or cisplatin, have shown modest activity in HRPC in combination with other agents (Fuse, 1996; Huan, 1999). This is unfortunate, since platinum agents and taxanes have been shown to be synergistic in pre-clinical (Rogers, 2002) and clinical studies in a variety of tumor types (Le Chevalier, 1998; McGuire, 1996; Vaughn, 1998). Clinical experience with a platinum and a taxane in combination in men with HRPC is quite limited, but somewhat better response have been reported (Smith, 2003; Oh, Halabi, 2003).

Cisplatin, the first platinum analogue, was approved for use in 1978 and is still widely used. The introduction of cisplatin was followed by carboplatin in 1989, and most recently by oxaliplatin. Treatment with platinum analogues is limited by their toxicity. Neurotoxicity and nephrotoxicity are the main dose-limiting toxicities observed following cisplatin treatment. Myelosuppression, which may be cumulative, is the most significant toxicity following carboplatin treatment. Peripheral neurotoxicity and cold sensitivity are well documented in patients treated with oxaliplatin.

Picoplatin (previously known as AMD473 and ZD0473) is a new-generation platinum agent that in preclinical studies has shown evidence of antitumor activity in several tumor types Like other platinum analogues, picoplatin causes cell death by formation of covalent cross-links in DNA that interfere with DNA replication and transcription, leading to cell death. It has shown evidence of a synergistic effect with other chemotherapy during in vitro studies, and an absence of neuro- and nephrotoxicity. Picoplatin appears to avoid the undesirable nephrotoxicity and neurotoxicity associated with earlier platinum analogues both in both pre-clinical studies and in Phase I and Phase II trials. Gelmon et al. (2004) reported that, for a group of 33 patients with solid tumors, the dose limiting toxicity (DLT) for picoplatin and docetaxel was 120 mg/m² and 75 mg/m², respectively, and the recommended Phase II dose (or “PTD”) is 100 mg/m² and 75 mg/m², respectively. They reported that one pre-treatment naïve prostate cancer patient achieved a PR at an unspecified dose.

The efficacy of platinum analogues is limited by several (intrinsic or acquired) mechanisms of resistance, including impaired cellular uptake, intracellular inactivation by thiols (e.g., reduced glutathione (GSH)), and enhanced DNA repair and/or increased tolerance to platinum-DNA adducts (Perez, 1998). Pre-clinical studies indicate that picoplatin can overcome these three mechanisms of resistance. This has been demonstrated in vitro and by using human ovarian xenografts tumor models that exhibit resistance to cisplatin (Beale, 2003; Raynaud, 1997; Holford, Raynaud, 1998; Holford, Sharp, 1998).

Picoplatin and processes for making picoplatin and for using picoplatin in treatment are disclosed and claimed in U.S. Pat. Nos. 5,665,771 (issued Sep. 9, 1997), and 6,518,428 (issued Feb. 11, 2003), and in PCT/GB0102060, filed May 10, 2001, published as WO2001/087313, which are incorporated herein by reference in their entireties.

Thus, a need exists for improved regimens for treatment of refractory cancers.

### SUMMARY OF THE INVENTION

An embodiment of the present invention provides a method of treatment of hormone refractory prostate cancer, comprising administering to a human patient afflicted with hormone refractory prostate cancer, the cancer being metastatic and chemotherapy-naive, substantially concurrently, picoplatin and docetaxel, wherein a dose of picoplatin of at least 120 mg/m² and a dose of docetaxel of about 60-100 mg/m² is administered intravenously at least once. The picoplatin and docetaxel can be administered at least twice, or can be administered about 2-12 times. The picoplatin and docetaxel can be administered at intervals of about 3-6 weeks.

In another embodiment of the invention, a method of treatment of hormone refractory prostate cancer, comprising administering to a human patient afflicted with hormone refractory prostate cancer, the cancer being metastatic and chemotherapy-naive, substantially concurrently, picoplatin and docetaxel, wherein the docetaxel is administered at a dosage of about 60-100 mg/m² and the picoplatin is administered at a dosage of about 120-180 mg/m² is provided

One embodiment of the invention comprises the administration of prednisone, the prednisone being administered to the patient orally at least once daily, e.g., twice daily. In one embodiment of the present method, the picoplatin and the docetaxel are both administered at intervals of about every three weeks, for example, 2 to 12 times (6 to 36 weeks), e.g., up to about ten times. The present method can extend the duration of life of the patient relative to the duration of life of a comparable patient not receiving the treatment, and can improve the quality of life of the patient relative to the quality of life of a comparable patient not receiving the treatment, and reduce the degree of pain and/or neurotoxicity, e.g., neuropathy, experienced by the patient relative to the degree of pain or neurotoxicity experienced by a comparable patient not receiving the treatment. The present method can also reduce the level of prostate-specific antigen of the patient relative to the level of prostate-specific antigen of a comparable patient not receiving the treatment, and thus act to stabilize the disease.

A further embodiment of the invention provides a method to reduce or eliminate the neurotoxicity of a neurotoxic anti-cancer drug by administering an effective amount, preferably an amount that is also a therapeutically-effective amount of picoplatin, as described herein, to a cancer patient who is being treated with the neurotoxic anti-cancer drug, such as a taxane and/or another Pt-containing drug.

The picoplatin and the docetaxel can each be administered at a dosage of about 60-120 mg/m², and about 60-100 mg/m², respectively, at each scheduled treatment, and the prednisone can be administered orally at a dosage of about 5 mg per dose. For example, the docetaxel can be administered intravenously in a dosage of about 60-75 mg/m² over a period of about 60 minutes, followed by intravenous administration of the picoplatin, up to about 30-60 min later, in a dosage of at least about 60 mg/m², e.g., up to about 100-200 mg/m², or at up to about 120-180 mg/m², over a period of about 1-2 hours. The prednisone can be administered orally twice daily in a dose of about 5 mg per administration. The present method can further comprise administration of a 5-HT₃ receptor antagonist, in an amount effective to control nausea and emesis.

In one embodiment, the docetaxel is given up to 3 hours, preferably at about 50±30 minutes prior to administration of picoplatin. For example, in one embodiment, the docetaxel is given up to about 1, 2 or 3 hours prior to administration of picoplatin. In another embodiment, the docetaxel is given up to about 10, 15, 20, 25, 30, 35, 40, 45, 50, 55, 60, 65, 70, 75, 80 or 85 minutes prior to picoplatin.

Picoplatin was found to have better tolerability than expected when combined with the higher doses of docetaxel in HRPC patients. Conversely, it was unexpected that conventionally employed doses of docetaxel could be administered in conjunction with effective doses of picoplatin in this range. For example, it has been found that the preferred treatment dose (PTD), is not necessarily the maximum tolerated dose (MTD), of picoplatin for use in the combination treatment with picoplatin and docetaxel in the first line treatment of chemotherapy naïve to RPC patients, in accord with the present method. The PTD preferably comprises about 120-150 mg/m² picoplatin and 75 mg/m² docetaxel, is administered every three weeks intravenously for at least about 6 cycles, preferably up to about 10 cycles of administration, providing a cumulative dose of up to about 1200-1500 mg/m² of picoplatin. The MTD is thus at least 120 mg/m² and may be higher. Prednisone can be administered at 5 mg twice daily during the period over which these cycles are carried out.

The present method also provides the administration of a dosage form for intravenous administration of picoplatin, comprising a solution comprising water, a tonicity adjuster, and about 0.5 mg/mL dissolved picoplatin.

### DETAILED DESCRIPTION OF THE INVENTION

Picoplatin, [SP-4-3]-ammine(dichloro)(2-methylpyridine)platinum(II), and pro-drugs thereof useful in the invention are disclosed in U.S. Pat. Nos. 5,669,771, 6,518,428 and 6,413,953, which are incorporated by reference herein. Picoplatin drug product is presented as a sterile, isotonic, aqueous solution for intravenous administration containing picoplatin a concentration of 0.5 mg/mL. The weight per mL is 1.005 g/mL. The composition is summarized on Table 1, below:

TABLE 1

| Ingredient | Function |
|---|---|
| Picoplatin | Active ingredient |
| Sodium Chloride USP | Tonicity adjuster |
| Water for Injection USP | Solvent |

Recent randomized trials have demonstrated that docetaxel (Taxotere®), when given with prednisone, leads to superior survival and improved rates of response in terms of pain, serum prostate-specific antigen (PSA) level, and quality of life (QOL). Based on these data, docetaxel 75 mg/m² has been approved by the FDA in combination with prednisone for use in the treatment of men with HRPC.

It is believed that cancer patients can be more effectively treated with the regimens of the present invention, which employ picoplatin instead of cisplatin, carboplatin or oxaliplatin, because they will experience fewer side effects, such as neuropathy, while preferably receiving higher doses of the platinum (Pt) drug.

Subjects with metastatic, chemotherapy-naive HRPC (documented progression of disease during adequate hormonal therapy), ECOG performance status of 0 or 1 and preserved organ function will undergo baseline assessment of disease. Subjects with radiographically documented metastatic disease that is either measurable (by RECIST criteria) or non-measurable disease will be eligible for study.

The MTD of picoplatin when administered with docetaxel every three weeks was determined. Subjects were initially treated with picoplatin plus docetaxel, 60 mg/m², given intravenously once every 3 weeks. Subjects received prednisone 5 mg orally twice daily.

The first cohort of subjects was treated with picoplatin at a dosage of 60 mg/m². The treatment was tolerated without dose limiting toxicity (DLT) in greater than ⅓ patients of the cohort, and the next cohort of subjects received picoplatin at 80 mg/m². Then additional cohorts received doses of picoplatin at increments of 20 mg/m² per dose level.

Picoplatin, 100 mg/m², plus docetaxel, 60 mg/m², were tolerated without DLT, and two additional separate cohorts were formed. Subjects in one of these two cohorts received 120 mg/m² picoplatin plus docetaxel, 60 mg/m². Subjects in the other of these two cohorts will receive picoplatin, 100 mg/m², plus docetaxel, 75 mg/m².

Unexpectedly DLT was observed in the 60 mg/m² docetaxel cohort but not in the 75 mg/m² docetaxel cohort. Therefore the PTD was selected to be picoplatin 120 mg/m² plus docetaxel 75 mg/m², and picoplatin will be increased until DLT is observed.

Depending on the pattern and severity of toxicity observed, additional intermediate picoplatin or docetaxel dose levels may be studied. The cohort size will be 3 subjects, to be expanded to 6 subjects if a DLT is observed. Within each cohort, one patient will be treated initially. If no DLT is observed within the following 3 weeks (1 drug cycle), the remaining two subjects may be treated. All three subjects within a cohort must have completed at least 1 cycle of the treatment regimen without a DLT prior to escalating the dose in the next cohort of subjects.

The term “MTD” is variously described in the art. In Gelmon (2004), MTD is defined as a level at which DLT occurs.

The maximum tolerated dose (MTD) herein will be defined as the dose of picoplatin below the dose at which at least one third of at least 6 subjects experience a DLT during the first cycle of treatment. Tolerance data from only the first cycle of treatment will be used for determination of the MTD. Additional subjects may be entered at any dosage level below the dose at which 2 of 6 patients have a DLT to obtain additional safety or efficacy data.

As the term is used herein, dose limiting toxicity (DLT) is defined as any of the following events occurring during the first cycle of therapy: For hematologic events: absolute neutrophil count (ANC) nadir <0.5×10⁹/L or platelet count <50×10⁹/L lasting for more than five days; or if ANC is <0.5×10⁹/L and colony stimulating factors are administered before 5 days; ANC <0.5×10⁹/L with fever (febrile neutropenia); or platelet count <25×10⁹/L. For non-hematologic events: any Grade 3 or 4 non-hematologic toxicity due to treatment, with the exception of alopecia, nausea or vomiting; grade 3 or 4 nausea or vomiting while receiving an optimal antiemetic regimen for prophylaxis and management; or treatment delays of greater than 3 weeks because of toxicity.

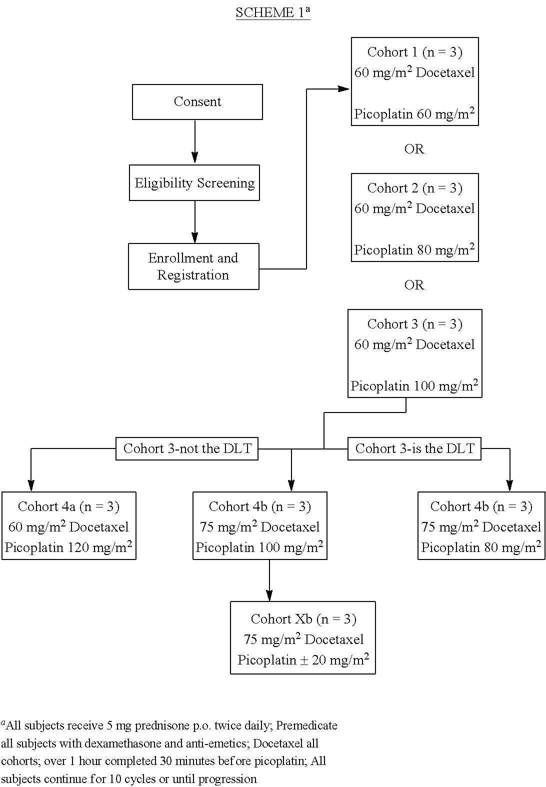

Evaluations will include assessment of adverse events (AEs), hematology values and PSA levels at the beginning of each treatment cycle. In addition, a CBC and platelet count are required once on Days 11-15 of Cycles 1 and 2 and during any cycle for which the doses of chemotherapy were reduced because of hematological toxicity. All patients will have serum chemistry tests measured every 6 weeks. Subjects with measurable disease will have imaging studies (CT scans) to determine the extent of disease every 6 weeks; those with non-measurable disease will be assessed every 12 weeks. All patients have bone scans every 12 weeks. Serum PSA will be measured 3 weeks after each chemotherapy infusion while receiving chemotherapy and then every 6 weeks until progression. Adverse events will be classified according to the Common Terminology Criteria Adverse Events of the National Cancer Institute (version 3).

This open-label Phase I/II study will allow the collection of further data about the tolerated dose of picoplatin in combination with prednisone and docetaxel in this patient population and of the safety and efficacy of picoplatin+docetaxel+prednisone, in preparation for a Phase III study.

The MTD of the combination therapy was previously defined in a Phase I study of patients with various malignancies. Those patients differed from the patient population in this study in that they were younger than typical patients with HRPC, most of them had received prior chemotherapy (0-8 cycles) and they did not all have bony metastases, typical of HRPC patients. Therefore a conservative approach was taken in the initial design of this study, i.e., the sole intent of the original study design was to confirm that the dose thought likely to be optimal in Part 2 (picoplatin, 100 mg/m², docetaxel, 60 mg/m²) would be safe and well tolerated in this patient population.

However, in view of the minimal toxicity in the first three cohorts, possibly because of the absence of any prior chemotherapy, the protocol has been modified to evaluate either a higher picoplatin dose or higher docetaxel dose to formally identify the MTD of these drugs in combination in this patient population. Different doses of picoplatin (e.g., 100 mg/m²) have been evaluated with 75 mg/m² docetaxel in addition to the ongoing evaluation of 60 mg/m².

Preferably the docetaxel is administered intravenously less than a few hours prior to the intravenous picoplatin, e.g., at 1-3 hours or simultaneously. The patients will receive 1-10 cycles of treatment, optimally 6-7 cycles of treatment with both drugs every 21 days.

Objective tumor responses will be defined by the RECIST system of unidimensional evaluation (Therasse, 2000). Responses will be confirmed at the next regularly scheduled evaluation, or by the disappearance of bone lesions on bone scans.

A preferred treatment dose (PTD), which for the purposes of the present application may or may not be the MTD, is found to be 120 mg/m² picoplatin with 75 mg/m² docetaxel, which can be administered in three-week cycles, for at least about six total cycles, up to about ten total cycles, providing a maximum cumulative dose of about 1200 mg/m². During this time period of 18-30 weeks, prednisone (5 mg) can be administered twice daily to the patients receiving the OTD program.

### Dose Reductions

Doses of picoplatin and docetaxel are delayed in the event of unresolved hematological toxicities. Doses of picoplatin and docetaxel are reduced in the event of hematological toxicity in the previous cycle, increased creatinine, or a change in body weight as described below. Once a subject has received a dose reduction, the doses of both picoplatin and docetaxel are not to be re-escalated. Subsequent treatments continue at that level unless the toxicity recurs, in which case a further reduction of the reduced doses is made. Up to two dose reductions are allowed. If an investigator determines that the degree of dose reduction should be greater than what is contained in these guidelines, investigator discretion takes precedence to protect the safety of the subject. Similarly, if an investigator determines that a dose reduction should be applied earlier than suggested by these guidelines, investigator discretion takes precedence to protect the safety of the subject.

The following hematological values are obtained before Day 1 chemotherapy for each cycle is administered: absolute neutrophil count (ANC) ≧1.5×10⁹/L; and platelet count ≧100×10⁹/L.

If these criteria are not met, then laboratory tests are carried out at a minimum of weekly intervals to see if the required laboratory values are reached. In the event of an absolute neutrophil count less than 0.5×10⁹/L or a platelet count less than 25×10⁹/L, hematology values are monitored at least twice weekly until the neutrophil and platelet counts have improved to above these levels.

A maximum of 21 days is allowed for resolution of the events that do not meet the dosing criteria (i.e., to Day 42 of the cycle). Subjects who do not meet the re-dosing criteria by Day 42 are withdrawn from further treatment for reasons of toxicity.

A dose-reduction of 20 mg/m² of picoplatin and 15 mg/m² of docetaxel is mandatory if any of the following hematological events are observed during the previous cycle: absolute neutrophil count <0.5×10⁹/L for at least 5 days; absolute neutrophil count <1.0×10⁹/L complicated with Grade ≧2 fever; platelet count <25×10⁹/L; not reaching a platelet count >100×10⁹/L by Day 21; or not reaching an absolute neutrophil count >1.5×109/L by Day 21.

Renal excretion is a major route of drug elimination. This fact suggests that impaired renal function may result in higher picoplatin or free-platinum serum concentrations and thus in greater myelotoxicity. Therefore, close attention is paid to serum creatinine and the need to modify the picoplatin dose in the event of renal function changes. Serum creatinine must be measured before every treatment. Dose modification for renal function is based upon serum creatinine, not calculated creatinine clearance. For subjects with abnormal serum creatinine, the dose of picoplatin (but not docetaxel) is modified according to Table 2:

TABLE 2

| Serum Creatinine | Dose Modification |
|---|---|
| ≦institutional ULN | recommended dose |
| >1.0 to 1.5 times ULN | reduce by 20 mg/m² |
| >1.5 to 2.0 times ULN | reduce by 40 mg/m^{2*} |
| >2.0 times ULN | discontinue treatment with picoplatin |

If dose reduction would result in the patient receiving <40 mg/m² of picoplatin, the patient should be taken off study treatment.

A change in weight of ≧10% from that used in the previous calculation of body surface area requires a recalculation in body surface area and appropriate modification of drug doses. Liver function tests (bilirubin, SGOT and SGPT) for liver function abnormalities must be measured every 6 weeks (or before every second dose of chemotherapy if treatment is delayed) and the dose of docetaxel administered modified according to the following table:

TABLE 3

| | SGOT/SGPT × | |
|---|---|---|
| Bilirubin × ULN | ULN | Docetaxel dose |
| <1 | <1.5 | 100% of previous dose |
| 1.1-1.5 | 1.6-2.5 | 80% of previous dose |
| 1.6-2.0 | 2.6-4.0 | 60% of previous dose |
| >2.0 | >4.0 | Omit |

If these criteria are not met, then these tests should be measured at a minimum of weekly intervals to see if the required laboratory values are reached. A maximum of 21 days is allowed for resolution of the events that do not meet dosing criteria (i.e., to Day 42 of the cycle). Subjects who do not meet re-dosing criteria by Day 42 must be withdrawn from further treatment for reasons of toxicity.

Non-hematological events (except nausea, vomiting, or alopecia), including treatment-related Grade 3 toxicity or any Grade 4 toxicity require dose modification of either or both picoplatin and docetaxel, or of prednisone, depending on the nature and clinical significance of the toxicity observed. In the event of uncertainty, the doses of picoplatin and docetaxel are omitted rather than given. In the event of different protocol-mandated dose modification(s), e.g., one for hematological and another for non-hematological toxicity, the doses of picoplatin and docetaxel are reduced by the greater of the recommended dose reductions, i.e., to the lower of the doses to be administered.

Subjects who do not meet the re-dosing criteria of absolute neutrophil count greater than or equal to 1.5×10⁹/L and platelet count greater than or equal to 100×10⁹/L by Day 42 (21 days after the intended day of retreatment) are withdrawn from further treatment for reasons of toxicity. Subjects requiring doses of picoplatin below 40 mg/m² according to the above criteria are removed from study.

### Quality of Life and Degree of Pain

Quality of life for prostate cancer patients is well known to include a variety of factors. For example, the article “Quality-of-life outcomes in men treated for localized prostate cancer”, by M. S. Litwin, R. D. Hays, A. Fink, P. A. Ganz, B. Leake, G. E. Leach and R. H. Brook, Journal of the American Medical Association, v. 273, no. 2, Jan. 11, 1995, provides various methods for assessing self-perceived quality of life among prostate cancer patients. As described therein, general health related quality of life (HRQOL) can be measured with the RAND 36-Item Health Survey 1.0. Cancer-specific HRQOL can be measured with the Cancer Rehabilitation Evaluation System-Short Form and the Functional Assessment of Cancer Therapy-General form. The article further describes an additional method for evaluation of HRQOL factors involving sexual, urinary, and bowel systems, which can all be involved in unpleasant prostate cancer symptoms. Issues including both “function” and “bother” are assessed. In “Long-Term Outcomes Among Localized Prostate Cancer Survivors: Health-Related Quality-of-Life Changes After Radical Prostatectomy, External Radiation, and Brachytherapy,” by David C. Miller, Martin G. Sanda, Rodney L. Dunn, James E. Montie, Hector Pimentel, Howard M. Sandler, William P. McLaughlin, John T. Wei, Journal of Clinical Oncology, v. 23, no. 12, 2772, Apr. 20, 2005, questionnaires assessing urinary irritative-obstructive, urinary incontinence, bowel, and sexual functions are used to evaluate HRQOL issues for men with prostate cancer. These established HRQOL assessments can be administered to patients at various times, and the changes in responses can be evaluated. The inventive method of treatment will provide more favorable HRQOL assessments by patients following administration of the picoplatin/docetaxel combination as described herein than the assessments obtained from the patients immediately prior to initiation of the therapy.

Similarly, the degree of pain experienced by prostate cancer patients can be assessed through questionnaires and self-evaluation by the patients. In “Quality of Life and Pain in Advanced Stage Prostate Cancer: Results of a Southwest Oncology Group Randomized Trial Comparing Docetaxel and Estramustine to Mitoxantrone and Prednisone,” by Donna L. Berry, Carol M. Moinpour, Caroline S. Jiang, Donna Pauler Ankerst, Daniel P. Petrylak, Lynne V. Vinson, Primo N. Lara, Sharon Jones, Mary E. Taplin, Patrick A. Burch, Maha H. A. Hussain, E. David Crawford, Journal of Clinical Oncology, v. 12, no. 18, 2828, Jun. 20, 2006, the assessment of pain and quality of life was carried out using the European Organisation for Research and Treatment of Cancer (EORTC) Quality of Life Questionnaire-Core30 (QLQ-C30), (Aaronson N K, Ahmedzai S, Bergman B, et al: The European Organization for Research and Treatment of Cancer QLQ-C30: A quality-of-life instrument for use in international clinical trials in oncology. J Natl Cancer Inst 85:365-376, 1993), the Prostate Cancer Module for the QLQ-C30 (PR-25), and the McGill Pain Questionnaire-Short Form (MPQ-SF) (Melzack R: The short-form McGill Pain Questionnaire. Pain 30:191-197, 1987). Specifically related to pain assessment, the MPQ-SF (Melzack R: The McGill pain questionnaire: From description to measurement. Anesthesiology 103:199-202, 2005) is a validated measure of pain including three sections: pain words, pain degree, and pain intensity. The pain words total score (0 to 45) is obtained for the rating of 15 words describing pain with a 0 to 3 response scale (none to severe). This score reflects the quality of experienced pain (eg, throbbing, aching, tender). A second score, adapted from the original visual analog scale, reflects the degree of pain on a 0 to 10 numerical scale (Wilkie D J, Lovejoy N, Dodd M, et al: Cancer pain intensity measurement: Concurrent validity of three instruments-finger dynamometer, pain intensity number scale, visual analogue scale. The Hospice Journal 6:1-13, 1990). The present pain intensity (PPI) pain item from the MPQ-SF, one of two primary QOL end points, lists responses ranging from 0 (no pain) to 5 (excruciating).

The inventive method of treatment will provide more favorable HRQOL assessments by patients following administration of the picoplatin/docetaxel combination as described herein than the assessments obtained from the patients immediately prior to initiation of the therapy. All the above references are incorporated herein by reference in their entireties.

### Definitions of PSA Response and Progression

PSA response is defined as a reduction from baseline of at least 50% maintained for at least 4 weeks).

PSA progression is defined as a confirmed increase from the nadir of either at least 25% for men with no PSA response or at least 50% for all others. The detailed criteria described by the PSA Working Group (Bubley, 1999) will be used: PSA progression is the time from the day treatment is initiated until the time the PSA has increased 50% above the nadir (in subjects who achieve at least a 50% decline in PSA and in whom the PSA has risen a minimum of 5 ng/mL) or, in subjects without a PSA decrease of 50%, the time at which a 25% increase in PSA has been achieved.

Confirmation of PSA progression by a second PSA obtained after an interval of at least one week must be obtained.

The duration of PSA response will be defined as the time between the first and last evaluations at which the response criterion was met.

### Time to Progression Objective Response and Progression-Free Survival (PFS)

TTP of objective response is the time from the day treatment is initiated until one of the following occurs: ≧20% increase in the sum of the longest diameter of target lesions from post-therapy nadir, clear worsening of nonmeasurable disease (e.g., bone scan), or appearance of new lesions).

Progression-free survival (PFS) is the time from initiation of treatment until objective disease progression, PSA progression, or death.

### Results

### 1. Phase 1.

As outlined in Scheme 1, picoplatin has been given to sequential cohorts of chemotherapy naïve subjects having HRPC at 60, 80, 100, and 120 mg/m² with docetaxel 60 mg/m², and picoplatin at 100 and 120 mg/m² with docetaxel 75 mg/m². 34 patients have been treated. Six patients enrolled at picoplatin at 120 mg/m² plus docetaxel at 60 mg/m². Nine enrolled at picoplatin 120 mg/m² plus docetaxel at 75 mg/m².

Therapy has been well tolerated. Dose-limiting toxicity (Grade 4 neutropenia) has been observed at 120 mg/m²+60 mg/m² docetaxel. Dose reduction for persistent hematological toxicity has been infrequent below 120 mg/m² picoplatin and there has been no cumulative myelotoxicity.

Picoplatin can be safely administered with docetaxel+prednisone in chemo-naïve patients at doses of up to 100-150 mg/m² with HRPC, e.g., preferably at 110-125 mg/m², i.e., 120 mg/m², using both 60 mg/m² and 75 mg/m² of docetaxel. No cumulative myelotoxicity was observed. The tolerance to the combination of 120 mg/m² picoplatin with 75 mg/m² docetaxel was unexpected in view of the confirmed toxicity of docetaxel.

Five patients of the 34 enrolled have expired, two from progressive disease. Three patients withdrew, ten patients completed ten cycles. A PSA response was observed in 19/32 subjects (59%). A 45% response would be expected with docetaxel alone. CT scans have identified partial responses in 2 of 15 evaluable patients, 25 patients have stable disease, (no progression observed by CT or bone scan) and four patients had progressive disease.

### 2. Phase II.

Thirty-two HRPC patients with progression of metastatic disease (PSA or radiographic) during hormonal therapy received up to ten cycles of 120 mg/m² of IV picoplatin, and docetaxel at 75 mg/m² Q3W with 5 mg oral prednisone bid. The demographics of the patient population are summarized on Table 4, below.

TABLE 4
Demographics (n = 32*)
Age (years)

| | |
|---|---|
| Median (range) | 67 (50-84) |

Age Group

| | |
|---|---|
| <65 years | 12 (38%) |
| ≧65 years | 20 (63%) |

ECOG PS

| | |
|---|---|
| 0 | 8 (25%) |
| 1 | 22 (69%) |
| Unknown | 2 (6%) |
| PSA, Median (range) | 499 (6-6682) |
| Measurable Disease, n (%) | 13 (41%) |

Type of Progression, n (%)

| | |
|---|---|
| PSA | 14 (44%) |
| Radiographic | 2 (6%) |
| Both | 16 (50%) |

Number of Sites of Metastatic Disease

| | |
|---|---|
| Median (range) | 2 (1-5) |

Sites of Metastatic Disease, n (%)

| | |
|---|---|
| Bone | 31 (97%) |
| Lymph Nodes (any location) | 11 (34%) |
| Lung | 6 (19%) |
| Liver | 5 (16%) |
| Other | 9 (28%) |

*intent to treat population

The picoplatin exposure is summarized in Table 5, below.

TABLE 5
Picoplatin Exposure (n = 29*)
Number of Cycles

| | |
|---|---|
| Total | 211 |
| Median | 10 |
| Range | 1-10 |

Picoplatin Cumulative Dose (mg/m²)

| | |
|---|---|
| Median | 900 |
| Range | 120-1200 |

Dose Reductions, n (%)

| | |
|---|---|
| Number of Patients | 12 (4%) |
| Number of Reductions | 21 (10%) |

Picoplatin Dose Intensity (mg/m²/3 weeks)

| | |
|---|---|
| Median | 116 |
| Range | 45-120 |

Docetaxel Dose Intensity (mg/m²/3 weeks)

| | |
|---|---|
| Median | 74 |
| Range | 30-75 |

*One patient received docetaxel only, no picoplatin.

Treatment was discontinued for toxicity or disease progression. The adverse events, hematological assessment and subject disposition, are summarized on Tables 6-8 below.

TABLE 6
Adverse Events (n = 30%)*

| | All Events** | Grade ¾ |
|---|---|---|
| Alopecia | 40% | NA |
| Asthenia | 27% | 7% |
| Serum creatinine increased*** | 23% | 0 |
| Nausea | 20% | 0 |
| Diarrhea | 17% | 3% |
| ALT Increased | 10% | 7% |
| AST Increased | 10% | 0 |
| Vomiting | 10% | 0 |
| Pyrexia | 7% | 3% |
| Hypersensitivity | 7% | 7% |
| Fatigue | 7% | 0 |
| Osteoarthritis | 7% | 0 |

*Patients who received any study drug
**Events that occurred in >1 patient
***When assessed and graded using lab values, 25% had grade 1 and 4% had grade 2. There were no grade 3 or 4 creatinine levels.
NA = not applicable.

TABLE 7
Hematologic Assessments (n = 28*)

| | Grade | N (%) |
|---|---|---|
| ANC** | 1 | 4 (14%) |
| | 2 | 6 (21%) |
| | 3 | 4 (14%) |
| | 4 | 9 (32%) |
| Platelets | 1 | 18 (64%) |
| | 2 | 6 (21%) |
| | 3 | 1 (4%) |
| | 4 | 1 (4%) |
| Hemoglobin | 1 | 9 (32%) |
| | 2 | 13 (64%) |
| | 3 | 4 (14%) |
| | 4 | 2 (7%) |

*Data not available for 1 patient.
**1 patient had grade 3 febrile neutropenia

TABLE 8
Subject Disposition (n = 32)

| | |
|---|---|
| Completed 10 cycles | 15 (47%) |
| Progressive Disease | 6 (19%) |
| Adverse Event | 8 (25%) |
| Subject Decision* | 2 (6%) |
| Death** | 1 (3%) |

*These 2 subjects received no study drug
**Not related to study drug

The PSA response was evaluated in 27 patients. Response is defined as a reduction from baseline of at least 50% maintained for at least 4 weeks, per Bubley criteria (JCO, 17:3461, 1999). A 78% response rate was observed. Radiologic response was evaluated using RECIST in patients with measurable disease. One partial response and 6 stable disease were observed in 12 patients with measurable disease (58% Disease Control). 1 patient with measurable disease was not treated. The overall response rate of 32 patients was 8%.

In conclusion, picoplatin was safely administered at 120 mg/m² Q3W in combination with full doses of docetaxel (75 mg/m²) and prednisone to patients with HRPC. Neutropenia was the main hematologic toxicity observed. In contrast to picoplatin monotherapy, thrombocytopenia was less severe and less frequent. No significant neurotoxicity was observed. This result is unexpected in view of the known neurotoxicity of docetaxel and of taxanes in general. Generally, the side effects are lower than would be expected from a combination of full-dose picoplatin and full-dose (75 mg/m²) docetaxel, it is believed that the picoplatin may be acting to counteract the neurotoxicity of the docetaxel and that this may be a general property of docetaxel, applicable to taxanes and to other classes of neurotoxic drugs, e.g., Pt-containing drugs.

### REFERENCES

All references below and other documents, patents and publications are incorporated by reference herein.
American Cancer Society Cancer Facts and Figures 2004. http://www.cancer.org/docroot/STT/stt_{—}0_{—}2004.asp?sitearea=STT&level=1
Beale et al., Br J Cancer 88(7):1128-1134, 2003.
Bubley et al., J Clin Oncol 17:3461-3467, 1999.
Canobbio et al., Eur J Cancer 29A(15):2094-96, 1993.
Cho et al., Urol Oncol 21:282-91, 2003.
Common Terminology Criteria for Adverse Events v3.0 (CTCAE). Publish Date Dec. 12, 2003. Web link: ctep.cancer.gov/forms/CTCAEv3.pdf.
Davidoff et al., N Eng J Med 13:825-6, 2001.
Douillard et al., Eur J Cancer 38(Suppl 8):525-S31, 2002.
Dowling et al., Ann Oncol 12:773-8, 2001.
Ernst et al., J Clin Oncol 21:3335-42, 2003.
Fuse et al., Int Urol Nephrol 28(1):79-85, 1996.
Gelmon et al., Ann Oncol 15(7):1115-22, 2004.
Gelmon et al., Ann Oncol 14:543-548, 2003.
Giaccone et al., Eur J Cancer 38(Suppl 8):519-S24, 2002.
Gore et al., Eur J Cancer 38(18):2416-2420, 2002.
Holford et al., Anticancer Drug Des 13:1-18, 1998.
Holford et al., Br J Cancer 77(3):366-373, 1998.
Huan et al., Am J Clin Oncol 22(5):471-474, 1999.
Kasamon et al., Curr Opin Urol 14:185-93, 2004.
Le Chevalier et al., Eur J Cancer 34:2032-2036, 1998.
Loening et al., J Urol 129(5):1001-6, 1983.
Mahler et al., Semin Surg Oncol 11:77-83, 1995.
McGuire et al., N Engl J Med 334:1-6, 1996.
Merrin et al., Urology 13:267-272, 1979.
Murakami et al., Eur J Cancer 38(Suppl 8):S1-S5, 2002.
Oh et al., Cancer 98:2592-8, 2003.
Oh et al., Cancer Medicine, 6th ed. Hamilton, Ontario: BC Decker; 1707-1740, 2003.
Oudard et al., J Clin Oncol 23(15):3343-51, 2005.
Perez, Eur J Cancer 34(10):1535-42, 1998.
Petrylak et al., N Engl J Med 351(15):1513-20, 2004.
Raynaud et al., Clin Cancer Res 3(11):2063-2074, 1997.
Reed, Cancer Principles and Practice of Oncology. Philadelphia, Pa.: JB Lippincott, 390-400, 1993.
Rini et al., Curr Treat Options Oncol 3:437-46, 2002.
Rogers et al., Eur J Cancer 38(12):1653-1660, 2002.
Rossof et al., Cancer Treat Rep 63(9-10):1557-1564, 1979.
Simon, Control Clin Trials 10(1):1-10, 1989.
Smith et al., Cancer 98:269-76, 2003.
Soloway et al., J Urol 129(1):56-61, 1983.
Tannock et al., N Engl J Med 351(15):1502-12, 2004.
Tannock et al., J Clin Oncol 14:1756-64, 1996.
Therasse et al., J Natl Cancer Inst 92(3):205-216, 2000.
Treat et al., Eur J Cancer 38(Suppl 8):513-18, 2002.
Vaughn et al., J Clin Oncol 16:255-260, 1998.

1. A method of treatment of hormone refractory prostate cancer, comprising:
administering to a human patient afflicted with hormone refractory prostate cancer, the cancer being metastatic and chemotherapy-naive, substantially concurrently, picoplatin and docetaxel, wherein a dosage of picoplatin of at least about 120 mg/m² and a dosage of docetaxel of about 60-100 mg/m² is administered intravenously at least once.

2. The method of claim 1 comprising administration of prednisone, the prednisone being administered to the patient orally at least once daily.

3. The method of claim 2 wherein the prednisone is administered twice daily.

4. The method of claim 1 wherein the picoplatin and the docetaxel are both administered about every 3-6 weeks.

5. The method of claim 1 wherein the duration of life of the patient is extended relative to the duration of life of a comparable patient not receiving the treatment.

6. The method of claim 1 wherein the quality of life of the patient is improved relative to the quality of life of the patient prior to the administration of the picoplatin and the docetaxel.

7. The method of claim 1 wherein the degree of pain felt by the patient is reduced relative to the degree of pain felt by the patient prior to the administration of the picoplatin and the docetaxel.

8. The method of claim 1 wherein the level of prostate-specific antigen of the patient is decreased relative to the level of prostate-specific antigen of a comparable patient not receiving the treatment.

9. The method of claim 1 wherein the picoplatin and the docetaxel are each administered at least twice

10. The method of claim 1 wherein the picoplatin and the docetaxel are each administered about 2 to 12 times.

11. The method of claim 10 wherein the picoplatin and the docetaxel are each administered 6 to 7 times.

12. A method of treatment of hormone refractory prostate cancer, comprising:
administering to a human patient afflicted with hormone refractory prostate cancer, the cancer being metastatic and chemotherapy-naive, substantially concurrently, picoplatin and docetaxel, wherein the docetaxel is administered at a dosage of about 60-100 mg/m² and the picoplatin is administered at a dosage of about 120-180 mg/m².

13. The method of claim 1 wherein a dose of at least 120 mg/m² picoplatin plus at least 75 mg/m² docetaxel is administered about every three to six weeks, for at least a total of about 6 cycles, optionally further comprising administering prednisone at a dose of about 5 mg once or twice daily.

14. The method of claim 13, wherein the dose is administered for up to a total of about 10 cycles.

15. The method of claim 2 wherein the prednisone is administered at a dosage of about 5 mg per dose.

16. The method of claim 1 wherein the docetaxel is administered intravenously in a dosage of about 60-75 mg/m² over a period of about 60 minutes, followed at up to about 30 minutes later by intravenous administration of the picoplatin over a period of about 1-2 hours.

17. The method of claim 1 wherein the docetaxel is administered at least about 75 mg/m² and picoplatin is administered at least about 120 mg/m².

18. The method of claim 1 wherein a subsequent dose of picoplatin is administered at about a 20 mg/m² lower dose than a previous dose.

19. The method of claim 1 wherein a subsequent dose of docetaxel is administered at about a 15 mg/m² lower dose than a previous dose.

20. The method of claim 1 further comprising administration of a 5-HT₃ receptor antagonist.

21. The method of claim 1, comprising administering the picoplatin in a dosage form comprising an isotonic solution comprising:
(a) water;
(b) a tonicity adjuster; and
(c) about 0.5 mg/mL dissolved picoplatin.

22. The method of claim 1 wherein the docetaxel is administered at about 75 mg/m² and the picoplatin is administered at about 120 mg/m².

23. A therapeutic method for reducing neurotoxicity comprising administering to a patient afflicted with cancer and treated with a neurotoxic anti-cancer drug, an amount of picoplatin effective to reduce to a grade of ≦2, preferably ≦1, or to eliminate, the neurotoxicity experienced by said patient, in the absence of said picoplatin.

24. The method of claim 23 wherein the patient is afflicted with hormone refractory prostate cancer.

25. The method of claim 23 wherein the neurotoxic anti-cancer drug is a taxane.

26. The method of claim 24 wherein the taxane is docetaxel.

27. The method of claim 26 wherein the patient is treated with about 60-75 mg/m² of docetaxel every 2-3 weeks.

28. The method of claim 26 wherein the patent is concurrently treated with about 110-130 mg/m² picoplatin.

29. The method of claim 28 wherein the patient is treated with about 120 mg/m² picoplatin.

30. The method of claim 29 wherein the picoplatin is administered prior to the administration of the docetaxel.

31. The method of claim 30 wherein the picoplatin is administered up to about 30 or up to about 60 minutes after administration of the docetaxel.

32. The method of claim 27 wherein about 2-10 3 week cycles of treatment are administered.

33. The method of claim 26 wherein is neurotoxicity is peripheral neuropathy.
